(19) United States
(12) Patent Application Publication
(10) Pub. No.: US 2013/0245250 A1
(43) Pub. Date: Sep. 19, 2013

(54) METHOD FOR PRODUCING L-FUCOSE

(71) Applicants: Andreas Schroven, Barssel (DE); Gyula Dekany, Queensland (AU); Christoph Röhrig, Muhlingen (DE); Ioannis Vrasidas, Thessaloniki (GR); Ignacio Figueroa Pérez, Miami, FL (US); Markus Hederos, Svedala (SE); Julien Boutet, La Plaine sur Mer (FR); Lars Kröger, Hamburg (DE); Piroska Kovács-Pénzes, Jaszbereny (HU); Ferenc Horváth, Pilisszentkereszt (HU); Christian Risinger, Rottweil (DE); Gergely Pipa, Budapest (HU)

(72) Inventors: Andreas Schroven, Barssel (DE); Gyula Dekany, Queensland (AU); Christoph Röhrig, Muhlingen (DE); Ioannis Vrasidas, Thessaloniki (GR); Ignacio Figueroa Pérez, Miami, FL (US); Markus Hederos, Svedala (SE); Julien Boutet, La Plaine sur Mer (FR); Lars Kröger, Hamburg (DE); Piroska Kovács-Pénzes, Jaszbereny (HU); Ferenc Horváth, Pilisszentkereszt (HU); Christian Risinger, Rottweil (DE); Gergely Pipa, Budapest (HU)

(73) Assignee: GLYCOM A/S, Lyngby (DK)

(21) Appl. No.: 13/878,739

(22) PCT Filed: Oct. 13, 2011
(86) PCT No.: PCT/EP2011/067906

§ 371 Date: May 28, 2013

(30) Foreign Application Priority Data
Oct. 14, 2010 (GB) 1017392.0

(51) Int. Cl. C07H 3/02

(52) U.S. Cl. CPC C07H 3/02
USPC 536/119; 536/124

(57) ABSTRACT

Method for producing L-fucose includes in a first aspect, a method for the preparation of L-fucose, wherein L-fucose precursors are produced from pectin and L-fucose is produced from the L-fucose precursors; in a second aspect, a method for the preparation of L-fucose from D-galacturonic acid or a salt thereof, wherein L-fucose precursors are produced from D-galacturonic acid of a salt thereof, and L-fucose is produced from the L-fucose precursors; and an L-fucose precursor as shown in Formula A, wherein R is a linear or branched chain saturated hydrocarbon group with 1-6 carbon atoms, such as methyl, ethyl, n-propyl, i-propyl, n-butyl, i-butyl, s-butyl, t-butyl, n-hexyl, etc., preferably a methyl group.

### FIELD OF THE INVENTION

The invention relates to a method for producing L-fucose starting from D-galacturonic acid or salts thereof, or from pectin or pectic substances.

### BACKGROUND OF THE INVENTION

Fucose (6-deoxy-galactose) is one of the examples of the so-called rare monosaccharides. Fucose is found in a wide variety of natural products from many different sources, in both D- and L-form. L-Fucose occurs in several human milk oligosaccharides, in the eggs of sea urchins and in frog spawn, and is present in polysaccharides from plants such as seaweed (in the form of fucoidan, sulphated fucose polymer), gum tragacanth, potato, kiwi fruit, soybean, winged bean varieties, canola, etc. In plant material, fucose is typically associated with plant polysaccharides, which are often highly branched structures having L-fucopyranosyl units either at the ends of or within the polysaccharide chains. Both N-and O-glycosyl chains of human or animal glycoproteins may contain L-fucose bound to the termini of the carbohydrate chains. Furthermore, extracellular polysaccharides from various bacteria, fungi and micro-algae also contain L-fucose.

Interest in L-fucose has recently increased because of its potential in the medical field in treating various disease conditions, such as tumors, inflammatory conditions and disorders relating to the human immune system. L-fucose has also applications in the cosmetic field, for instance as a skin moisturizing, skin regenerating and anti-aging agent or for prevention of epidermal (skin) inflammation.

Although enzyme- or microbe-assisted production of fucose is known from the art, L-fucose is usually obtained from natural sources or produced via chemical modifications of common monosaccharides (see a review on L-fucose: P. T. Vanhooren et al. J. Chem. Technol. Biotechnol. 74, 479 (1999) and references cited therein).

Regarding fucose production from natural sources, fucose containing oligosaccharides that can be isolated from biomass, preferably from algae e.g. by extraction, are hydrolyzed to provide a complex mixture containing fucose as well as related sugars and/or derivatives thereof. Recovery of fucose from the mixture typically needs sophisticated separation techniques such as treatment or chromatography with anion or cation exchange resins, dialysis, fractional crystallization, etc., depending on the nature of the accompanying sugars or sugar-related compounds.

With regard to chemical synthesis of fucose, chemical modifications of common monosaccharides have been published. Deoxygenation of the C-6 carbon of D-galactose results in D-fucose; however, this methodology is not practical for the synthesis of L-fucose as L-galactose is not available in quantity. L-fucose is obtainable from L-arabinose via a complex reaction sequence involving numerous intermediates. Inversion of configuration at C-5 and deoxygenation of C-6 in D-glucose provides L-fucose in a multistep procedure. Starting from D-mannose, a stereoselective chain elongation on C-1 and cleavage of the terminal glycol portion are needed to produce L-fucose. L-rhamnose as a 6-deoxy hexose requires OH-inversions, namely at C-2 and C-4 to yield L-fucose. Hitherto, D-galactose has seemed to be the most suitable starting material for producing L-fucose as there is no need to perform inversion: reduction of the C-1 formyl group and oxidation of the C-6 primary hydroxyl to formyl provides L-fucose. The common characteristic of the above-mentioned processes is the unavoidable temporary protection of the hydroxyls that are not to undergo the configurational inversion, deoxygenation, reduction and/or oxidation steps of the process. The numerous protection/deprotection steps, frequently requiring selective techniques, make these methodologies lengthy and cumbersome. In addition, in some cases laborious chromatographic separations are required to isolate intermediates from by-products.

The drawbacks mentioned above prevent elaborating large-scale manufacture of L-fucose. Thus there is still a vast need to provide alternative synthetic routes towards L-fucose that may enhance scale-up opportunities and facilitate low cost methodologies.

### SUMMARY OF THE INVENTION

The present invention provides in a first aspect a method for the preparation of L-fucose, wherein L-fucose precursors are produced from pectin and L-fucose is produced from the L-fucose precursors. In a second aspect, the present invention provides a method for the preparation of L-fucose from D-galacturonic acid or a salt thereof, wherein L-fucose precursors are produced from D-galacturonic acid or a salt thereof, and L-fucose is produced from the L-fucose precursors. An L-fucose precursor is also provided.

### DETAILED DESCRIPTION OF THE INVENTION

The chemical synthesis of organic compounds generally follows multistep synthetic pathways utilising protection and deprotection strategies. Preparing intermediates suitably armed with masking groups and removing them after the desired chemical transformation(s) require technological time and often isolation/purification efforts which prolong the whole synthetic sequence and raise the costs.

The present inventors provide a short synthetic route towards L-fucose that starts from readily available D-galacturonic acid or a salt thereof, or from the readily available pectin (also referred to as “pectins” or “pectic substances”), in which the requirement for OH-protection is reduced compared with prior art processes. In a preferred embodiment, no OH-protection is used during the process. Additionally, the intermediates used are preferably crystalline materials. Crystallization or recrystallization is one of the simplest and cheapest methods to isolate a product from a reaction mixture, separate it from contaminations and obtain the pure substance. Isolation or purification that uses crystallization makes the whole technological process robust and cost-effective, thus it is advantageous and attractive compared to other procedures. Further, the process can be conducted on a large scale efficiently, raising the possibility of commercially viable production of L-fucose.

The first aspect of the present invention provides a method for preparation of L-fucose from pectin comprising the steps of:

a) production of L-fucose precursors from pectin, and
b) production of L-fucose (compound 1) from the L-fucose precursors.

The term “L-fucose precursor” in the first aspect of the invention means intermediate compounds between pectin and L-fucose in the reaction sequence. For example, in Scheme 1 compound 5 and salts thereof, compound 4, compound 4′ and salts thereof, and compounds 3, 3′ and 2 each are L-fucose precursors. Further, in Scheme 2, compounds 6, 7 and 8 each are L-fucose precursors. An L-fucose precursor can be converted into another L-fucose precursor. Each conversion step (a) and (b) may comprise at least one synthetic step, in which the compounds formed may or may not be isolated before proceeding to a subsequent synthetic step.

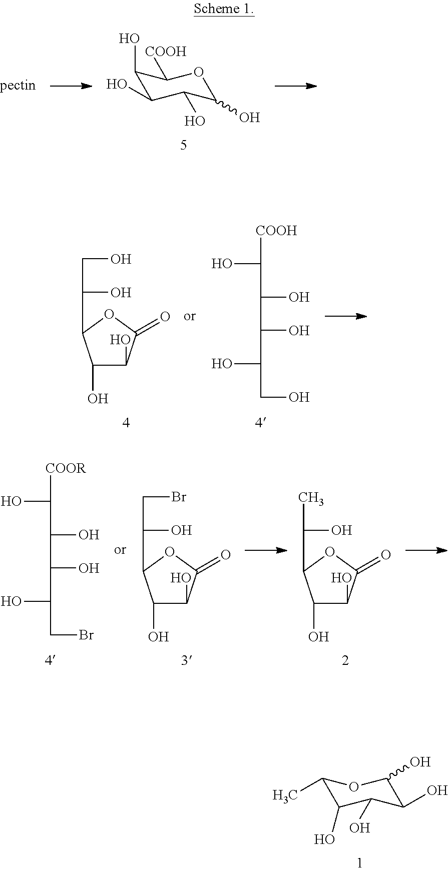

In one embodiment pectin is hydrolyzed to D-galacturonic acid (compound 5) or a salt thereof as an L-fucose precursor.

In an embodiment, D-galacturonic acid (compound 5) or a salt thereof is an L-fucose precursor in the method of the invention. Thus, the method comprises production of D-galacturonic acid (compound 5) or a salt thereof from pectin and production of L-fucose from D-galacturonic acid (compound 5) or a salt thereof. Preferably, the production of D-galacturonic acid (compound 5) or a salt thereof is carried out by hydrolysis of pectin.

In an embodiment, L-galactonic acid γ-lactone (compound 4) is an L-fucose precursor in the method of the invention. Thus, the method comprises the production of L-galactonic acid γ-lactone (compound 4) from pectin and the production of L-fucose from L-galactonic acid γ-lactone (compound 4). Preferably, the method comprises production of D-galacturonic acid or a salt thereof from pectin, production of L-galactonic acid γ-lactone (compound 4) from D-galacturonic acid or a salt thereof, and production of L-fucose from L-galactonic acid γ-lactone (compound 4).

In an embodiment, L-galactonic acid (compound 4′) or a salt thereof is an L-fucose precursor in the method of the invention. Thus, the method comprises the production of L-galactonic acid (compound 4′) or a salt thereof from pectin and the production of L-fucose from L-galactonic acid (compound 4′) or a salt thereof. Preferably, the method comprises production of D-galacturonic acid or a salt thereof from pectin, production of L-galactonic acid (compound 4′) or a salt thereof from D-galacturonic acid or a salt thereof, and production of L-fucose from L-galactonic acid (compound 4′) or a salt thereof.

In an embodiment, 6-bromo-6-deoxy-L-galactonic acid alkyl ester (compound 3) is an L-fucose precursor in the method of the invention. Thus, the method comprises the production of 6-bromo-6-deoxy-L-galactonic acid alkyl ester (compound 3) from pectin and the production of L-fucose from 6-bromo-6-deoxy-L-galactonic acid alkyl ester (compound 3). Preferably, the method comprises production of D-galacturonic acid or a salt thereof from pectin, production of 6-bromo-6-deoxy-L-galactonic acid alkyl ester (compound 3) from D-galacturonic acid or a salt thereof, and production of L-fucose from 6-bromo-6-deoxy-L-galactonic acid alkyl ester (compound 3). Preferably, the method comprises production of L-galactonic acid γ-lactone (compound 4), L-galactonic acid (compound 4′) or a salt thereof from pectin, production of 6-bromo-6-deoxy-L-galactonic acid alkyl ester (compound 3) from L-galactonic acid γ-lactone (compound 4), L-galactonic acid (compound 4′) or a salt thereof, and production of L-fucose from 6-bromo-6-deoxy-L-galactonic acid alkyl ester (compound 3). Preferably, the method comprises production of D-galacturonic acid or a salt thereof from pectin, production of L-galactonic acid γ-lactone (compound 4), L-galactonic acid (compound 4′) or a salt thereof from D-galacturonic acid or a salt thereof, production of 6-bromo-6-deoxy-L-galactonic acid alkyl ester (compound 3) from L-galactonic acid γ-lactone (compound 4), L-galactonic acid (compound 4′) or a salt thereof, and production of L-fucose from 6-bromo-6-deoxy-L-galactonic acid alkyl ester (compound 3). Preferably, the alkyl ester in compound 3 is a methyl ester.

In an embodiment, 6-bromo-6-deoxy-L-galactonolactone (compound 3′) is an L-fucose precursor in the method of the invention. Thus, the method comprises the production of 6-bromo-6-deoxy-L-galactonolactone (compound 3′) from pectin and the production of L-fucose from 6-bromo-6-deoxy-L-galactonolactone (compound 3′). Preferably, the method comprises production of D-galacturonic acid or a salt thereof from pectin, production of 6-bromo-6-deoxy-L-galactonolactone (compound 3′) from D-galacturonic acid or a salt thereof, and production of L-fucose from 6-bromo-6-deoxy-L-galactonolactone (compound 3′). Preferably, the method comprises production of L-galactonic acid γ-lactone (compound 4), L-galactonic acid (compound 4′) or a salt thereof from pectin, production of 6-bromo-6-deoxy-L-galactonolactone (compound 3′) from L-galactonic acid γ-lactone (compound 4), L-galactonic acid (compound 4′) or a salt thereof, and production of L-fucose from 6-bromo-6-deoxy-L-galactonolactone (compound 3′). Preferably, the method comprises production of D-galacturonic acid or a salt thereof from pectin, production of L-galactonic acid γ-lactone (compound 4), L-galactonic acid (compound 4′) or a salt thereof from D-galacturonic acid or a salt thereof, production of 6-bromo-6-deoxy-L-galactonolactone (compound 3′) from L-galactonic acid γ-lactone (compound 4), L-galactonic acid (compound 4′) or a salt thereof, and production of L-fucose from 6-bromo-6-deoxy-L-galactonolactone (compound 3′).

In an embodiment, L-fuconolactone (compound 2) is an L-fucose precursor in the method of the invention. Thus, the method comprises the production of L-fuconolactone (compound 2) from pectin and the production of L-fucose from L-fuconolactone (compound 2). Preferably, the method comprises production of D-galacturonic acid or a salt thereof from pectin, production of L-fuconolactone (compound 2) from D-galacturonic acid or a salt thereof, and production of L-fucose from L-fuconolactone (compound 2). Preferably, the method comprises production of L-galactonic acid γ-lactone (compound 4), L-galactonic acid (compound 4′) or a salt thereof from pectin, production of L-fuconolactone (compound 2) from L-galactonic acid γ-lactone (compound 4), L-galactonic acid (compound 4′) or a salt thereof, and production of L-fucose from L-fuconolactone (compound 2). Preferably, the method comprises production of 6-bromo-6-deoxy-L-galactonolactone (compound 3′) or 6-bromo-6-deoxy-L-galactonic acid alkyl ester (compound 3) from pectin, production of L-fuconolactone (compound 2) from 6-bromo-6-deoxy-L-galactonolactone (compound 3′) or 6-bromo-6-deoxy-L-galactonic acid alkyl ester (compound 3), and production of L-fucose from L-fuconolactone (compound 2). Preferably, the method comprises production of D-galacturonic acid or a salt thereof from pectin, production of L-galactonic acid γ-lactone (compound 4), L-galactonic acid (compound 4′) or a salt thereof from D-galacturonic acid or a salt thereof, production of L-fuconolactone (compound 2) from L-galactonic acid γ-lactone (compound 4), L-galactonic acid (compound 4′) or a salt thereof, and production of L-fucose from L-fuconolactone (compound 2). Preferably, the method comprises production of D-galacturonic acid or a salt thereof from pectin, production of 6-bromo-6-deoxy-L-galactonolactone (compound 3′) or 6-bromo-6-deoxy-L-galactonic acid alkyl ester (compound 3) from D-galacturonic acid or a salt thereof, production of L-fuconolactone (compound 2) from 6-bromo-6-deoxy-L-galactonolactone (compound 3′) or 6-bromo-6-deoxy-L-galactonic acid alkyl ester (compound 3), and production of L-fucose from L-fuconolactone (compound 2). Preferably, the method comprises production of L-galactonic acid γ-lactone (compound 4), L-galactonic acid (compound 4′) or a salt thereof from pectin, production of 6-bromo-6-deoxy-L-galactonolactone (compound 3′) or 6-bromo-6-deoxy-L-galactonic acid alkyl ester (compound 3) from L-galactonic acid γ-lactone (compound 4), L-galactonic acid (compound 4′) or a salt thereof, production of L-fuconolactone (compound 2) from 6-bromo-6-deoxy-L-galactonolactone (compound 3′) or 6-bromo-6-deoxy-L-galactonic acid alkyl ester (compound 3), and production of L-fucose from L-fuconolactone (compound 2). Preferably, the method comprises production of D-galacturonic acid or a salt thereof from pectin, production of L-galactonic acid γ-lactone (compound 4), L-galactonic acid (compound 4′) or a salt thereof from D-galacturonic acid or a salt thereof, production of 6-bromo-6-deoxy-L-galactonolactone (compound 3′) or 6-bromo-6-deoxy-L-galactonic acid alkyl ester (compound 3) from L-galactonic acid γ-lactone (compound 4), L-galactonic acid (compound 4′) or a salt thereof, production of L-fuconolactone (compound 2) from 6-bromo-6-deoxy-L-galactonolactone (compound 3′) or 6-bromo-6-deoxy-L-galactonic acid alkyl ester (compound 3), and production of L-fucose from L-fuconolactone (compound 2).

Preferred conditions and reagents for carrying out the transformations above are given in the description following the second aspect of the invention.

The second aspect of the invention provides a method of producing L-fucose from D-galacturonic acid or a salt thereof. Preferably, the method comprises the steps of:

a) producing one or more L-fucose precursors from D-galacturonic acid or a salt thereof, and
b) producing L-fucose from the one or more L-fucose precursors.

Similarly to the first aspect of the invention, the term “L-fucose precursor” in the second aspect of the invention means intermediate compounds between D-galacturonic acid or a salt thereof and L-fucose in the reaction sequence. For example, in Scheme 1, compound 4, compound 4′ and salts thereof, and compounds 3, 3′ and 2 each are L-fucose precursors in the method of the second aspect of the invention. Further, in Scheme 2, compounds 6, 7 and 8 each are L-fucose precursors. An L-fucose precursor can be converted into another L-fucose precursor. Each conversion step (a) and (b) may comprise at least one synthetic step, in which the compounds formed may or may not be isolated before proceeding to a subsequent synthetic step.

In an embodiment, L-galactonic acid γ-lactone (compound 4) is an L-fucose precursor in the method of the second aspect of the invention. Thus, the method comprises the production of L-galactonic acid γ-lactone (compound 4) from D-galacturonic acid or a salt thereof and the production of L-fucose from L-galactonic acid γ-lactone (compound 4).

In an embodiment, L-galactonic acid (compound 4′) or a salt thereof is an L-fucose precursor in the method of the second aspect of the invention. Thus, the method comprises the production of L-galactonic acid (compound 4′) or a salt thereof from D-galacturonic acid or a salt thereof and the production of L-fucose from L-galactonic acid (compound 4′) or a salt thereof.

In an embodiment, 6-bromo-6-deoxy-L-galactonic acid alkyl ester (compound 3) is an L-fucose precursor in the method of the second aspect of the invention. Thus, the method comprises the production of 6-bromo-6-deoxy-L-galactonic acid alkyl ester (compound 3) from D-galacturonic acid or a salt thereof and the production of L-fucose from 6-bromo-6-deoxy-L-galactonic acid alkyl ester (compound 3). Preferably, the method comprises production of L-galactonic acid γ-lactone (compound 4), L-galactonic acid (compound 4′) or a salt thereof from D-galacturonic acid or a salt thereof, production of 6-bromo-6-deoxy-L-galactonic acid alkyl ester (compound 3) from L-galactonic acid γ-lactone (compound 4), L-galactonic acid (compound 4′) or a salt thereof, and production of L-fucose from 6-bromo-6-deoxy-L-galactonic acid alkyl ester (compound 3). Preferably, the alkyl ester in compound 3 is a methyl ester.

In an embodiment, 6-bromo-6-deoxy-L-galactonolactone (compound 3′) is an L-fucose precursor in the method of the second aspect of the invention. Thus, the method comprises the production of 6-bromo-6-deoxy-L-galactonolactone (compound 3′) from D-galacturonic acid or a salt thereof and the production of L-fucose from 6-bromo-6-deoxy-L-galactonolactone (compound 3′). Preferably, the method comprises production of L-galactonic acid γ-lactone (compound 4), L-galactonic acid (compound 4′) or a salt thereof from D-galacturonic acid or a salt thereof, production of 6-bromo-6-deoxy-L-galactonolactone (compound 3′) from L-galactonic acid γ-lactone (compound 4), L-galactonic acid (compound 4′) or a salt thereof, and production of L-fucose from 6-bromo-6-deoxy-L-galactonolactone (compound 3′).

In an embodiment, L-fuconolactone (compound 2) is an L-fucose precursor in the method of the second aspect of the invention. Thus, the method comprises the production of L-fuconolactone (compound 2) from D-galacturonic acid or a salt thereof and the production of L-fucose from L-fuconolactone (compound 2). Preferably, the method comprises production of L-galactonic acid γ-lactone (compound 4), L-galactonic acid (compound 4′) or a salt thereof from D-galacturonic acid or a salt thereof, production of L-fuconolactone (compound 2) from L-galactonic acid γ-lactone (compound 4), L-galactonic acid (compound 4′) or a salt thereof, and production of L-fucose from L-fuconolactone (compound 2). Preferably, the method comprises production of 6-bromo-6-deoxy-L-galactonolactone (compound 3′) or 6-bromo-6-deoxy-L-galactonic acid alkyl ester (compound 3) from D-galacturonic acid or a salt thereof, production of L-fuconolactone (compound 2) from 6-bromo-6-deoxy-L-galactonolactone (compound 3′) or 6-bromo-6-deoxy-L-galactonic acid alkyl ester (compound 3), and production of L-fucose from L-fuconolactone (compound 2). Preferably, the method comprises production of L-galactonic acid γ-lactone (compound 4), L-galactonic acid (compound 4′) or a salt thereof from D-galacturonic acid or a salt thereof, production of 6-bromo-6-deoxy-L-galactonolactone (compound 3′) or 6-bromo-6-deoxy-L-galactonic acid alkyl ester (compound 3) from L-galactonic acid γ-lactone (compound 4), L-galactonic acid (compound 4′) or a salt thereof, production of L-fuconolactone (compound 2) from 6-bromo-6-deoxy-L-galactonolactone (compound 3′) or 6-bromo-6-deoxy-L-galactonic acid alkyl ester (compound 3), and production of L-fucose from L-fuconolactone (compound 2).

Preferred conditions and reagents for carrying out the transformations above in both the first and second aspects of the invention are given in the following description.

Pectins (also referred to as “pectin” or “pectic substances”) are complex polysaccharides found in the primary cell walls and intercellular regions of higher plants, and contain linear chains of 1,4-linked α-D-galactopyranuronic acid residues. The galacturonic acid monomer may be substituted by neutral monosaccharides, mainly by D-xylose and/or D-apiose. A group of pectins called rhamnogalacturonan contain a repeating disaccharide of α-D-galacturonic acid-(1→2)-α-L-rhamnose from which neutral sugars like D-galactose, L-arabinose and D-xylose may branch off. The majority of carboxylic groups are present as the methyl ester, and the remaining carboxylic acid groups are present as their salts, in particular salts of Na, K or Ca, or as the free acids. Oranges and citrus-like fruits contain quite large amount of pectins, with a lesser amount being found in fruits and vegetables such as apples, apricots, gooseberries, carrots, quinces, guavas, plums, cherries, grapes, strawberries, etc.

Pectic substances can be hydrolyzed to D-galacturonic acid by means of acids or enzymes. During hydrolysis the interglycosidic linkages, and any ester groups that are present, are cleaved. In acidic hydrolysis, strong aqueous inorganic and organic acids may be used, such as hydrochloric acid, sulfuric acid, trifluoroacetic acid, etc., and the hydrolysis is conducted typically at a temperature between 70° C. and reflux. The insoluble materials are removed by filtration; filter aid materials such as kieselguhr, supercel or activated carbon may be added to help the filtration of gelatinous residues. After neutralization, D-galacturonic acid is precipitated or crystallized out as the acid or in the form of an acid addition salt such as the sodium salt, calcium salt, potassium salt, barium salt or sodium calcium double salt. In enzymatic hydrolysis, any pectinase or pectin lyase with pectolytic, hemicellulolytic and carbohydratase activity can be applied. Typical hydrolysis methods are described in e.g. S. Morell et al. J. Biol. Chem. 105, 15 (1934), S. Fukunaga et al. Bull. Chem. Soc. Japan 13, 272 (1938), U.S. Pat. No. 2,338,534, WO 02/42484 or H. Garna et al. Food Chem. 96, 477 (2006) and references cited therein.

In another embodiment D-galacturonic acid or salts thereof as an L-fucose precursor is reduced to L-galactonic acid (compound 4′) or salt thereof or its γ-lactone (compound 4) as another L-fucose precursor.

Generally, reductive agents like Na/Hg and tetrahydroborate salts or Raney Ni in H₂ atmosphere are suitable for reducing the formyl group of a uronic acid to hydroxyl while the carboxylic acid portion remains intact. Of course, it is preferred not to use sodium amalgam due to the toxicity of the mercury, the difficulty in handling the amalgam safely and the difficulty in disposing of the reagent responsibly. This is particularly true when conducting reactions on a large scale.

The chemoselectivity of the reducing agents described above ensures the formation of aldonic acids, thus D-galacturonic acid (compound 5) or salts thereof can be converted to L-galactonic acid (4′). The free galactonic acid can be isolated from the aqueous solution with evaporation under vacuum at low temperature or in the form of a salt. In solution, L-galactonic acid (4′) converts spontaneously to γ-lactone (4). The process may be facilitated by raising the temperature.

In a preferred embodiment, a D-galacturonate salt, preferably the sodium or calcium salt, is treated with a tetraborohydride salt as reductive agent. The borohydride used can be any commercially available borohydride such as sodium, lithium, potassium, calcium, zinc or aluminium tetraborohydride, L-, K-, S-, KS- or LS-selectride, sodium cyanoborohydride, sodium or lithium triethylborohydride, etc., preferably sodium, lithium, potassium, calcium or aluminium tetraborohydride, most preferably sodium tetraborohydride. The resulting L-galactonic acid derivative can be used either as the pure compound or as the crude reaction product in the next step.

In another embodiment L-galactonic acid (4′), a salt thereof, or its γ-lactone (4) is converted to 6-bromo-6-deoxy-L-galactonic acid alkyl ester (general formula 3) or 6-bromo-6-deoxy-L-galactonolactone (compound 3′) with regioselective bromination.

The regioselective bromination means a bromine-hydroxyl exchange in the primary position of a vicinal diol portion containing derivative in a bromo-de-hydroxylation reaction.

A typical reagent for the introduction of a bromine atom into a primary position is hydrogen bromide/acetic acid (HBr/AcOH). The reaction can be conducted at room temperature or with gentle heating up to 45-50° C. Under the conditions used, partial or full acetylation of the secondary hydroxyls also takes place, and the acetyl groups can be removed by addition of C₁₋₆-alkyl alcohol to the reaction mixture giving rise to 6-bromo-6-deoxy-L-galactonolactone (compound 3′). All of galactonic acid (4′)/a salt thereof, and galactonolactone (4) give the same compound 3′ under these conditions. Upon prolonged (more than 12 h) alcoholysis the lactone ring opens and the carboxyl is esterified, yielding the corresponding 6-bromo-6-deoxy-L-galactonic acid alkyl ester (general formula 3), wherein the alkyl group is a linear or branched chain saturated hydrocarbon group with 1-6 carbon atoms, such as methyl, ethyl, n-propyl, i-propyl, n-butyl, i-butyl, s-butyl, t-butyl, n-hexyl, etc. Another method of conducting bromine/hydroxyl exchange would be treatment of compound 4 with PPh₃/CBr₄ in the presence of a base like pyridine, triethyl amine, Hünig's base, etc. In this case, as no alcohol is present, the product would be the lactone 3′.

In a preferred embodiment L-galactonolactone is treated with HBr/AcOH followed by prolonged methanolysis to give methyl 6-bromo-6-deoxy-L-galactonate (general formula 3, wherein R is methyl). Preferably, the starting material is the crude residue from the reaction of sodium D-galacturonate with NaBH₄.

In another embodiment, 6-bromo-6-deoxy-L-galactonic acid alkyl ester (general formula 3) or 6-bromo-6-deoxy-L-galactonolactone (compound 3′) as L-fucose precursor is converted to L-fuconolactone (compound 2) as another L-fucose precursor by debromination with catalytic hydrogenolysis.

The term “catalytic hydrogenolysis” here refers to reduction with hydrogen (whether provided as the gas, generated in situ, or otherwise), wherein the bromine atom is exchanged with a hydrogen atom, in the presence of a catalyst. Typically, the reaction takes place in a protic solvent or in a mixture of protic solvents. A protic solvent may be selected from a group consisting of water, acetic acid or C₁-C₆ alcohol. A mixture of one or more protic solvents with one or more appropriate aprotic organic solvents miscible partially or fully with the protic solvent(s) (such as THF, dioxane, ethyl acetate, acetone, etc.) may also be applied. Water, one or more C₁-C₆ alcohols or a mixture of water and one or more C₁-C₆ alcohols are preferably used as solvent system. The reaction mixture may comprise a solution or a suspension of the carbohydrate in the solvent or mixture of solvents, at any suitable concentration. The reaction mixture is stirred at a temperature in the range of 10-100° C., preferably between 25-70° C., in a hydrogen atmosphere of 1-50 bar in the presence of a catalyst such as palladium, Raney nickel or any other appropriate metal catalyst, preferably palladium on charcoal (Pd—C) or palladium black, until the completion of the reaction is reached. Transfer hydrogenation may also be performed, when the hydrogen is generated in situ from cyclohexene, cyclohexadiene, formic acid or ammonium formate. Organic or inorganic bases and/or basic ion exchange resins can also be used to improve the kinetics of the hydrogenolysis. Preferred organic bases include but are not limited to tertiary amines such as triethylamine, diisopropyl ethylamine (Hünig's base), and pyridine, etc. Preferred basic ion exchange resins are those having quaternary amino groups.

In a preferred embodiment methyl 6-bromo-6-deoxy-L-galactonate (general formula 3, wherein R is methyl) is debrominated in methanol under hydrogen atmosphere in the presence of Pd—C.

In another embodiment L-fuconolactone (compound 2) as an L-fucose precursor is converted to L-fucose (compound 1).

It has been reported that, when unprotected fuconolactone is treated with Na/Hg, only a moderate yield of fucose can be achieved (S. Akiya et al. Yakugaku Zasshi 74, 1296 (1954), Chem. Abstr. 49, 83987 (1955)). We speculate that this may be because of its high ability for overreduction to fucitol. The production of a significant quantity of alditol beside the desired aldose seems to be unavoidable. In addition, the use of toxic and potentially dangerous reducing agents such as sodium amalgam is not preferred in modern laboratories, particularly when working on a larger scale, as discussed above. It should also be noted that when a synthetic product is intended for human consumption, contamination with even trace quantities of mercury is to be avoided.

A recent paper reports on the unsuccessful attempts to reduce of fuconolactone to fucose with a range of reducing agents (J. M. Gardiner et al. Synlett 2685 (2005)). This report suggests that reduction of unprotected compound 2 to compound 1 is not possible. Indeed, the authors of another recent paper chose to protect the secondary alcohols of fuconolactone with acetyl groups and to use a somewhat unusual reducing agent in order to reduce the protected fuconolactone to a protected lactone that when deprotected gave L-fucose (see Binch et al., Carbohydrate Res 306, 409 (1998)).

The present inventors have surprisingly found that borohydrides are able to reduce fuconolactone to fucose. In addition, an acceptable proportion of L-fucose is present in the reaction mixture along with the by-product L-fucitol, and so the yield of L-fucose obtained is acceptable and an improvement on prior art processes, while avoiding the use of toxic and difficult to handle reducing agents such as sodium amalgam.

According to a preferred embodiment, L-fuconolactone (compound 2) as an L-fucose precursor is reduced to L-fucose (compound 1) with a borohydride salt. The borohydride used can be any commercially available borohydride such as sodium, lithium, potassium, calcium, zinc or aluminium tetraborohydride, L-, K-, S-, KS- or LS-selectride, sodium cyanoborohydride, sodium or lithium triethylborohydride, etc., preferably sodium lithium, potassium, calcium or aluminium tetraborohydride, most preferably sodium tetraborohydride. The reduction is conducted in aqueous acidic medium, preferably between pH 3-5, which can be maintained by continuous addition of an acid or with the presence of acidic cation exchange resin and/or using an acidic buffer system. Nevertheless, whatever conditions are chosen, the formation of L-fucitol is always detectable. As both compounds are crystalline, they can be separated by means of fractional crystallization or chromatography.

In a further preferred method L-fucitol, separated out as by-product, can be used as further L-fucose precursor in order to raise the efficiency of the L-fucose production. L-fucitol may be converted to L-fucose by the following method: isopropylidenation of L-fucitol (compound 6) to 2,3:4,5-di-O-isopropylidene-L-fucitol (compound 7), oxidation to di-O-isopropylidene-L-fucose (compound 8) and deprotection to L-fucose (compound 1, see Scheme 2).

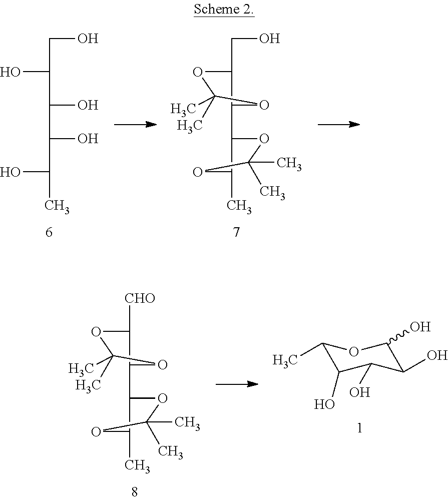

Isopropylidenation of L-fucitol can take place in acetone (being the reagent and also the solvent) in the presence of a soluble acid (practically all kinds of organic and inorganic acids are suitable, the most frequently used ones are sulfuric acid, HCl and p-toluenesulfonic acid) or insoluble acid (e.g. ion exchange resins in H⁺ form). A Lewis acid (e.g. zinc chloride, stannous chloride, titanium chloride, boron trifluoride etherate, etc.) as catalyst can also be of preference. Transacetalation with dimethoxy propane under acid catalysis can also be employed.

Oxidation of the primary hydroxyl in compound 7 to formyl can be conducted with strong oxidizing agents such as chromium(VI) reagents (CrO₃-pyridine complex, Jones reagent, PCC, pyridinium dichromate, trimethylsilyl chromate), MnO₂, KMnO₄, RuO₄, CAN, or DMSO in combination with one of DCC, Ac₂O, oxalyl chloride, tosyl chloride, bromine, chlorine, etc., in a known manner. A preferred oxidising agent combination for conducting this oxidation is trichloroisocyanuric acid and TEMPO.

The isopropylidene groups in compound 8 can be removed by acidic hydrolysis. Water (as well as being the reagent) may serve as solvent as well. The acids used are generally protic acids selected from but not limited to acetic acid, trifluoroacetic acid, HCl, formic acid, sulphuric acid, perchloric acid, oxalic acid, p-toluenesulfonic acid, benzenesulfonic acid, cation exchange resins, etc., which may be present in from catalytic amount to large excess. The hydrolysis may be conducted at temperatures between 20° C. and reflux until completion of the reaction is reached, generally a couple of hours, depending on temperature, concentration and pH. Preferably, organic acids including but not limited to aqueous solutions of acetic acid, formic acid, chloroacetic acid, oxalic acid, cation exchange resins, etc. are used at a temperature in the range of 40-75° C.

In another preferred embodiment, L-fuconolactone (compound 2) as an L-fucose precursor is converted to L-fucose (compound 1) in a method comprising the steps of: protection of L-fuconolactone secondary hydroxyls to give compounds of general formula 9, reduction of the protected L-fuconolactone derivative to protected L-fucofuranose (compounds of general formula 10), and deprotection to L-fucose (see Scheme 3). The protection can be effected by means of acylation, silylation, acetal or ether formation. The range of possible reducing agents, beside the ones already mentioned at the direct partial reduction, can be broadened to include selective reducing agents that are not suitable for use in a protic medium, such as boranes (e.g. disiamylborane) and aluminium hydrides (e.g. diisobutyl aluminium hydride (dibal)). The resulting protected fucose derivative with free 1-OH can then be deprotected by known methods to fucose.

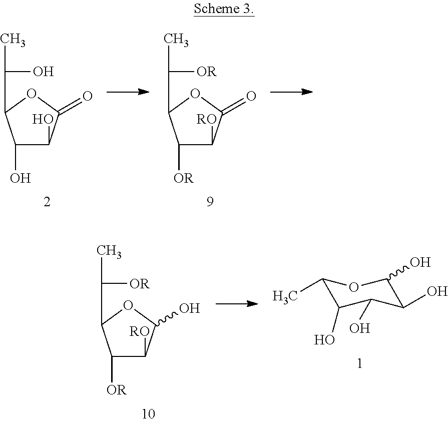

In a further aspect of the present invention is provided 6-bromo-6-deoxy-L-galactonic acid alkyl esters (compound 3) wherein alkyl means a linear or branched chain saturated hydrocarbon group with 1-6 carbon atoms, such as methyl, ethyl, n-propyl, i-propyl, n-butyl, i-butyl, s-butyl, t-butyl, n-hexyl, etc. In a preferred embodiment alkyl is methyl.

Other features of the invention will become apparent in the course of the following descriptions of exemplary embodiments which are given for illustration of the invention and are not to be limiting thereof.

### Examples

### 1. Methyl 6-bromo-6-deoxy-L-galactonate (General Formula 3, R=methyl)

A) Sodium D-galacturonate (5, sodium salt) (4.00 g, 18.5 mmol) was dissolved in water (40 mL) and cooled to 0° C. While being stirred a freshly prepared 0.5 M aqueous solution of NaBH₄ (20.0 mL) was added dropwise to the reaction mixture. The solution was stirred at 4° C. overnight. Amberlite IR 120 (H⁺) (approx. 4.0 g) was added and the solution was evaporated. The residue was taken up in MeOH (20 mL) and evaporated again after removal of the resin by filtration. The residue was taken up in 33% HBr/AcOH (6.00 mL) and the reaction mixture was stirred overnight at 45° C. MeOH (20 mL) and activated carbon were added to the stirred solution at this temperature. The activated carbon was filtered out and the solution was stirred at 45° C. overnight. The reaction mixture was allowed to cool to room temperature while the product began to crystallize. The suspension was stirred for 1 h at 0° C. The product was filtered to yield 2.05 g (7.54 mmol, 41%) of product.

B) L-Galactono-1,4-lactone (compound 4, 4.00 g, 22.5 mmol) was dissolved in 33% HBr/AcOH (6.00 mL) and stirred for 9 h at 45° C. Methanol (20.0 mL) was added dropwise at this temperature. The reaction mixture was stirred for another 16 h at 45° C. The product began to crystallize after 1 h. The product was filtered out and washed with cold methanol to yield 4.21 g (15.5 mmol, 69%) of colourless crystals.

¹H NMR (DMSO-d₆, 300 MHz): δ=3.35-3.34 (m, 1H), 3.90-3.85 (m, 1H), 3.79-3.76 (d, 1H, J=9.6), 3.64-3.49 (m, 5H), 3.38-3.35 (m, 1H).

¹³C NMR (DMSO-d₆, 75 MHz): δ=174.3, 71.7, 70.5, 69.5, 68.9, 51.5, 35.9.

### 2. L-Fucono-1,4-lactone (2)

Methyl 6-bromo-6-deoxy-L-galactonate (3) (3.60 g, 13.2 mmol) was suspended with Amberlite IRA-67 (5.60 g) and 10% Pd/C (500 mg) in MeOH (50.0 mL). The reaction mixture was stirred overnight at 70° C. under 10 bar of H₂ pressure. The catalyst was filtered off and the reaction mixture was evaporated. The residue was dissolved in water (appr. 30 mL) and Amberlite IR 120 (H⁺) was added. The mixture was evaporated and the residue was taken up again in the same volume of water. The water was evaporated and the procedure was repeated again. The resin was filtered off and the water was evaporated. The crude material crystallized to yield 1.85 g (11.4 mmol, 87%).

¹H NMR (D₂O, 300 MHz): δ=4.56 (d, 1H, J=8.8 Hz), 4.23-4.18 (m, 1H), 4.11-4.07 (m, 1H), 4.00-3.92 (m, 1H), 1.26 (d, 3H, J=6.6 Hz).

¹³C NMR (D₂O, 75 MHz): δ=176.1, 83.8, 74.0, 73.4, 65.8, 18.0.

### 3. L-Fucose (1)

L-Fuconolactone (2) (1.00 g, 6.17 mmol) was dissolved in aqueous boronic acid buffer (50 mL) and cooled to 0° C. Amberlite IR 120 (H⁺) (approx. 50 mL) was added to the stirred solution. Freshly prepared aqueous NaBH₄ (3.00 g NaBH₄ in 150 mL water) was added dropwise in three portions to the stirred solution at 0° C. The pH was controlled to be between 4 and 5. After addition of NaBH₄ the reaction mixture was evaporated and the crude product mixture was dissolved in hot EtOH. The solution was allowed to warm to room temperature. The product mixture started to crystallize during the warming of the solution. The resulting suspension was stirred overnight at 4° C. and the crystals were filtered off to yield 720 mg of a mixture of L-fucitol and L-fucose (6:4).

### 4. L-Fucitol (6)

A mixture of L-fucitol/L-fucose (1:1, 800 mg) was dissolved in water (50 mL) and cooled to 0° C. NaBH₄ (500 mg) was added to the stirred solution at this temperature. The reaction mixture was stirred for 1.5 h at 0° C. then acidified by addition of Amberlite IR 120 (H⁺). The water was evaporated and the residue was taken up in MeOH and evaporated three times. Before the last evaporation the resin was filtered off. The product was crystallized from MeOH to yield 700 mg (4.22 mmol).

¹H NMR (CDCl₃, 300 MHz): δ=4.08-4.01 (m, 1H), 3.94-3.89 (m, 1H), 3.64-3.57 (m, 2H), 3.45-3.41 (m, 1H), 1.19 (d, 3H, J=6.6 Hz).

¹³C NMR (CDCl₃, 300 MHz): δ=72.9, 70.5, 69.8, 66.1, 63.3, 18.7.

### 5. 2,3:4,5-Di-O-isopropylidene-L-fucitol (7)

L-Fucitol (1.00 g, 6.02 mmol) was suspended in acetone (10 mL). Sulfuric acid (0.23 mL) was added dropwise to the stirred reaction mixture. The solution was stirred for 1 h at room temperature. The solution was neutralized by addition of Et₃N (1.76 mL) and evaporated. The residue was taken up in DCM (50 mL) and washed twice with water (30 mL), 1M HCl (30 mL), sat. NaHCO₃ (30 mL) and once with brine (20 mL), dried over MgSO₄ and evaporated. The residue was dissolved in hot heptane (15 mL) and stored overnight at 4° C. The crystals were filtered off giving 935 mg (3.80 mmol, 63%) of product.

¹H NMR (C₆D₆, 300 MHz): δ=4.07-3.95 (m, 2H), 3.83-3.73 (m, 3H), 3.46-3.41 (m, 1H), 1.36-1.21 (m, 15H).

¹³C NMR (C₆D₆, 300 MHz): δ=109.4, 109.0, 83.2, 81.8, 79.3, 77.2, 62.8, 27.4, 27.0, 26.8, 26.6, 18.4.

### 6. Di-O-isopropylidene-L-fucose (8)

Trichloroisocyanuric acid (4.71 g, 20.3 mmol) was added to a stirred solution of 2,3:4,5-di-O-isopropylidene-L-fucitol (5.00 g, 20.3 mmol) in DCM (50 mL) and the mixture was cooled to 0° C. TEMPO (33.0 mg, 202 μmol, 1%) was added and the cooling bath was removed to allow the reaction mixture to warm to room temperature. The reaction was complete after 20 min. The mixture was diluted with DCM (50 mL) and washed with sat. NaHCO₃ (40 mL), 1 M aqueous HCl (40 mL) and twice with brine (30 mL). The organic phase was dried over MgSO₄ and evaporated to yield 4.09 g (16.8 mmol, 82%) of product as colourless crystals.

¹H NMR (C₆D₆, 300 MHz): δ=9.49 (d, 1H), 4.33-4.29 (m, 1H), 4.04-4.00 (m, 1H), 3.95-3.91 (m, 1H), 3.55-3.50 (m, 1H), 1.34-1.15 (m, 15H).

¹³C NMR (C₆D₆, 300 MHz): δ=197.4, 110.0, 107.3, 81.7, 80.9, 76.4, 74.3, 25.8, 25.3, 25.1, 24.6, 16.6.

### 7. L-Fucose (1)

Di-O-isopropylidene-L-fucose (780 mg, 3.20 mmol) was suspended in water (6.0 mL) and Amberlite IR 120 (H⁺) (appr. 1 mL) is added. The reaction mixture was stirred for 2 h at 60° C. The resin was filtered off and the water was evaporated. The residue was dissolved in hot EtOH (2.0 mL), the solution was allowed to cool to room temperature and some seeding crystals were added. The solution was stirred for 30 min at 4° C. The crystals formed were filtered off and washed with cold EtOH to yield 410 mg (2.50 mmol, 78%) of colourless crystals.

1. A method of producing L-fucose from D-galacturonic acid or a salt thereof, comprising:
a) producing at least one L-fucose precursor from D-galacturonic acid or a salt thereof, and
b) producing L-fucose from the at least one L-fucose precursor.

2. The method according to claim 1, comprising:
a) producing L-galactonic acid, a salt thereof or L-galactonic acid γ-lactone from D-galacturonic acid or a salt thereof, and
b) producing L-fucose from L-galactonic acid, a salt thereof, or L-galactonic acid γ-lactone.

3. The method according to claim 2, wherein the production of L-galactonic acid, a salt thereof or its γ-lactone from D-galacturonic acid or a salt thereof comprises the treatment of D-galacturonic acid or a D-galacturonate salt with NaBH₄.

4. The method according to claim 1, comprising:
a) producing 6-bromo-6-deoxy-L-galactonic acid alkyl ester or 6-bromo-6-deoxy-L-galactonolactone from D-galacturonic acid or a salt thereof, and
b) producing L-fucose from 6-bromo-6-deoxy-L-galactonic acid alkyl ester or 6-bromo-6-deoxy-L-galactonolactone.

5. The method according to claim 4, wherein a) comprises producing L-galactonic acid, a salt thereof or its γ-lactone from D-galacturonic acid or a salt thereof and producing 6-bromo-6-deoxy-L-galactonic acid alkyl ester or 6-bromo-6-deoxy-L-galactonolactone from L-galactonic acid, a salt thereof or its γ-lactone.

6. The method of claim 5, wherein the production of 6-bromo-6-deoxy-L-galactonic acid alkyl ester or 6-bromo-6-deoxy-L-galactonolactone from L-galactonic acid, a salt thereof or its γ-lactone comprises regioselective bromination.

7. The method according to claim 5, wherein the production of 6-bromo-6-deoxy-L-galactonic acid alkyl ester from L-galactonic acid γ-lactone comprises the treatment of L-galactonolactone with HBr/AcOH followed by prolonged alkanolysis to give 6-bromo-6-deoxy-L-galactonic acid alkyl ester.

8. The method according to claim 1, comprising:
a) producing L-fuconolactone from D-galacturonic acid or a salt thereof, and
b) producing L-fucose from L-fuconolactone.

9. The method according to claim 8, wherein a) comprises producing 6-bromo-6-deoxy-L-galactonic acid alkyl ester or 6-bromo-6-deoxy-L-galactonolactone from D-galacturonic acid or a salt thereof, and producing L-fuconolactone from 6-bromo-6-deoxy-L-galactonic acid alkyl ester or 6-bromo-6-deoxy-L-galactonolactone.

10. The method of claim 9, wherein the production of L-fuconolactone from 6-bromo-6-deoxy-L-galactonic acid alkyl ester or 6-bromo-6-deoxy-L-galactonolactone comprises debromination with catalytic hydrogenolysis.

11. The method according to claim 10, wherein methyl 6-bromo-6-deoxy-L-galactonate is reduced with Pd—C/H₂.

12. The method according to claim 1, wherein b) comprises producing L-fucose from L-fuconolactone.

13. The method according to claim 8, wherein L-fuconolactone is reduced to L-fucose with a borohydride salt, preferably with sodium tetrahydroborate.

14. The method according to claim 8, wherein the production of L-fucose from L-fuconolactone comprises:
protection of fuconolactone secondary hydroxyls,
reduction of the protected fuconolactone derivative to protected fucose, and
deprotection of the protected fucose to give fucose.

15. A method according to claim 1, comprising the hydrolysis of pectin to produce D-galacturonic acid or salts thereof.

16. A method for preparation of L-fucose from pectin, comprising:
a) producing at least one L-fucose precursor from pectin, and
b) producing L-fucose from the at least one L-fucose precursor.

17. The method according to claim 16, comprising:
a) hydrolysis of pectin to produce D-galacturonic acid or salts thereof as an L-fucose precursor, and
b) producing L-fucose from D-galacturonic acid or a salt thereof.

18. The method according to claim 16, comprising:
a) producing L-galactonic acid, salts thereof, or L-galactonic acid γ-lactone from pectin, and
b) producing L-fucose from L-galactonic acid, a salt thereof, or L-galactonic acid γ-lactone.

19. The method according to claim 17, wherein a) comprises producing D-galacturonic acid or salts thereof from pectin and producing L-galactonic acid, salts thereof or its γ-lactone from D-galacturonic acid or salts thereof.

20. The method according to claim 19, wherein the production of L-galactonic acid, salts thereof or its γ-lactone from D-galacturonic acid or salts thereof comprises the treatment of a D-galacturonate salt with NaBH₄.

21. The method according to claim 16, comprising:
a) producing 6-bromo-6-deoxy-L-galactonic acid alkyl ester or 6-bromo-6-deoxy-L-galactonolactone from pectin, and
b) producing L-fucose from 6-bromo-6-deoxy-L-galactonic acid alkyl ester or 6-bromo-6-deoxy-L-galactonolactone.

22. The method according to claim 21, wherein a) comprises producing L-galactonic acid, salts thereof or its γ-lactone from pectin and producing 6-bromo-6-deoxy-L-galactonic acid alkyl ester or 6-bromo-6-deoxy-L-galactonolactone from L-galactonic acid, salts thereof or its γ-lactone.

23. The method of claim 22, wherein producing 6-bromo-6-deoxy-L-galactonic acid alkyl ester or 6-bromo-6-deoxy-L-galactonolactone from L-galactonic acid, salts thereof or its γ-lactone comprises regioselective bromination.

24. The method according to claim 22, wherein producing 6-bromo-6-deoxy-L-galactonic acid alkyl ester from L-galactonic acid γ-lactone comprises the treatment of L-galactonolactone with HBr/AcOH followed by prolonged alkanolysis to give 6-bromo-6-deoxy-L-galactonic acid alkyl ester.

25. The method according to claim 16, comprising:
a) producing L-fuconolactone from pectin, and
b) producing L-fucose from L-fuconolactone.

26. The method according to claim 25, wherein a) comprises producing 6-bromo-6-deoxy-L-galactonic acid alkyl ester or 6-bromo-6-deoxy-L-galactonolactone from pectin, and producing L-fuconolactone from 6-bromo-6-deoxy-L-galactonic acid alkyl ester or 6-bromo-6-deoxy-L-galactonolactone.

27. The method of claim 26, wherein producing L-fuconolactone from 6-bromo-6-deoxy-L-galactonic acid alkyl ester or 6-bromo-6-deoxy-L-galactonolactone comprises debromination with catalytic hydrogenolysis.

28. The method according to claim 27, wherein methyl 6-bromo-6-deoxy-L-galactonate is reduced with Pd—C/H₂.

29. The method according to claim 16, wherein b) comprises the production of L-fucose from L-fuconolactone.

30. The method according to claim 29, wherein L-fuconolactone is reduced to L-fucose with a borohydride salt, preferably with sodium tetrahydroborate.

31. The method according to claim 29, wherein the production of L-fucose from L-fuconolactone comprises:
protection of fuconolactone secondary hydroxyls,
reduction of the protected fuconolactone derivative to protected fucose, and
deprotection of the protected fucose to give fucose.

32. The method according to claim 1, wherein L-fucitol is an L-fucose precursor, and b) further comprises:
isopropylidenation of L-fucitol to 2,3:4,5-di-O-isopropylidene-L-fucitol, oxidation of 2,3:4,5-di-O-isopropylidene-L-fucitol to di-O-isopropylidene-L-fucose, and
deprotection of di-O-isopropylidene-L-fucose to L-fucose.

33. A compound as shown in Formula A below, wherein R is a linear or branched chain saturated hydrocarbon group with 1-6 carbon atoms, such as methyl, ethyl, n-propyl, i-propyl, n-butyl, i-butyl, s-butyl, t-butyl, n-hexyl, etc., preferably a methyl group.

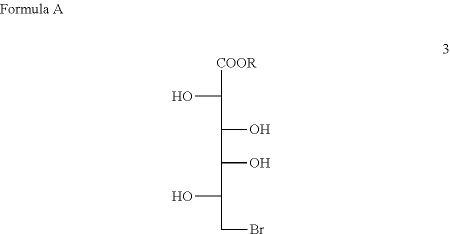

34. The method according to claim 16, wherein L-fucitol is an L-fucose precursor, and (b) further comprises:
isopropylidenation of L-fucitol to 2,3:4,5-di-O-isopropylidene-L-fucitol, oxidation of 2,3:4,5-di-O-isopropylidene-L-fucitol to di-O-isopropylidene-L-fucose, and
deprotection of di-O-isopropylidene-L-fucose to L-fucose.
